(19) United States
(12) Patent Application Publication
(10) Pub. No.: US 2018/0305292 A1
(43) Pub. Date: Oct. 25, 2018

(54) DERIVATIVES OF NONSTEROIDAL ANTI-INFLAMMATORY DRUGS

(71) Applicant: Wolfgang ALBRECHT, Ulm (DE)

(72) Inventors: Wolfgang ALBRECHT, Ulm (DE); Roland SELIG, Ulm (DE); Richard GUSERLE, Kötz (DE)

(21) Appl. No.: 15/769,028

(22) PCT Filed: Oct. 20, 2016
(86) PCT No.: PCT/EP2016/075247

§ 371 Date: Apr. 17, 2018

(30) Foreign Application Priority Data
Oct. 21, 2015 (EP) 15190881.1

(51) Int. Cl. C07C 69/593; C07C 69/618; C07D 487/04; A61P 25/00

(52) U.S. Cl. CPC C07C 69/593; A61P 25/00; C07D 487/04; C07C 69/618

(57) ABSTRACT

The present invention relates to novel compounds, e.g. for use as a medicament. In particular, the present invention relates to novel derivatives of certain nonsteroidal anti-inflammatory drugs, suitable as a medicament, preferably in the treatment and/or prevention of systemic diseases, autoimmune diseases, and/or inflammatory diseases, for example osteoarthritis, rheumatoid arthritis, multiple sclerosis and psoriasis. Further, the invention relates to a pharmaceutical composition comprising the novel compounds.

The present invention relates to novel compounds, e.g. for use as a medicament. In particular, the present invention relates to novel derivatives of certain nonsteroidal anti-inflammatory drugs, suitable as a medicament, preferably in the treatment and/or prevention of systemic diseases, autoimmune diseases, and/or inflammatory diseases, for example osteoarthritis, rheumatoid arthritis, multiple sclerosis and psoriasis. Further, the invention relates to a pharmaceutical composition comprising the novel compounds.

Nonsteroidal anti-inflammatory drugs, often abbreviated to NSAIDs, are drugs that provide several effects, such as analgesic (pain-killing) and antipyretic (fever-reducing) effects. Further, in higher doses, NSAIDs exhibit also an anti-inflammatory effect. Michael Fine “Quantifying the Impact of NSAID-Associated Events”, The American Journal Of Managed Cure, Vol. 19, no. 14, November 2013, pages 267-272 reports that “nonsteroidal anti-inflammatory drugs (NSAIDs) are the ornerstone of pain management. Compared to steroids or glucocorticosteroids, which also exhibit analgesic and anti-inflammatory effects, nonsteroidal anti-inflammatory drugs bear the advantage that they are non-narcotic and are thus a non-addictive treatment.

Nonsteroidal anti-inflammatory drugs can for example be classified by their chemical structures. Among NSAIDs there are the classes of those bearing a free acid group. These comprise for example salicylates, such as acetylsalicylic acid, propionic acid derivatives, such as (RS)-2-(4-(2-methylpropyl)phenyl)propanoic acid, (+)-(S)-2-(6-methoxynaphthalen-2-yl)propanoic acid or (RS)-2-(2-fluorobiphenyl-4-yl)propanoic acid, acetic acid derivatives, such as 2-{1-[(4-chlorophenyl)carbonyl]-5-methoxy-2-methyl-1H-indol-3-yl}acetic acid or 2-(2-(2,6-dichlorophenylamino)phenyl)acetic acid, anthranilic acid derivatives, such as 2-(2,3-dimethylphenyl)aminobenzoic acid or 2-{[3-(trifluoromethyl)phenyl]amino}benzoic acid, or others, such as [6-(4-chlorophenyl)-2,2-dimethyl-7-phenyl-2,3-dihydro-1H-pyrrolizin-5-yl]acetic acid.

The mechanism of action of NSAIDs is reported to be based on the nonselective inhibition of the activity of COX-1 (cyclooxygenase-1) and COX-2 (cyclooxygenase-2), wherein the inhibition is reversible.

As indicated above, in order to reach an appropriate anti-inflammatory effect the nonsteroidal anti-inflammatory drug has to be administered in high doses.

However, the use of high-dosed NSAIDs might cause undesired side-effects. For example, high-dosed NSAIDs, in particular when taken over a longer period, are reported to increase the risk of a myocardial infarction and stroke or to cause erectile dysfunction. Further, and more frequently, adverse effects such as an increased risk of kidney problems or in particular gastrointestinal (GI) problems are brought into interrelation with the administration of high-dosed NSAIDs. In fact, the main adverse drug reactions associated with NSAID use relate to direct and indirect irritation of the gastrointestinal (GI) tract. Again, Michael Fine “Quantifying the Impact of NSAID-Associated Events”, The American Journal Of Managed Cure, Vol. 19, no. 14, November 2013, pages, 267-272 states that “most of the NSAIDs were associated with a relative risk for upper gastrointestinal complications between 2 and 4. Researchers also found that the risk was dose-dependent. NSAIDs cause a dual assault on the GI tract: direct irritation of the gastric mucosa by acidic molecules and inhibition of COX-1 and COX-2 which reduces the levels of protective prostaglandins. The latter is reported to cause increased gastric acid secretion, diminished bicarbonate secretion, diminished mucus secretion and diminished trophic effects on epithelial mucosa. Common gastrointestinal adverse drug reactions can be nausea/vomiting, dyspepsia gastric ulceration/bleeding and diarrhea.

Thus, there is still a need for compounds with improved properties, preferably when being used as medicament, in particular in the treatment and/or prevention of systemic diseases, autoimmune diseases, and/or inflammatory diseases.

The compounds of the present invention should be capable of being applied in doses high enough to show an appropriate anti-inflammatory effect. Further, the undesired side-effects associated with common NSAIDs should be reduced.

Hence, it was an object of the present invention to overcome the drawbacks of the above-mentioned available nonsteroidal anti-inflammatory drugs.

In particular it was an object to develop compounds to be used as a medicament for the above-mentioned diseases, wherein said compounds show an appropriate anti-inflammatory effect and wherein said compounds do not show undesired side-effects such as gastrointestinal problems or kidney problems.

### SUMMARY OF THE INVENTION

According to the present invention, the above objectives are achieved by the specific compounds described herein by Formula (I). Said compounds can be used as a medicament for the treatment and/or prevention of systemic diseases, autoimmune diseases, and/or inflammatory diseases, for example osteoarthritis, rheumatoid arthritis, multiple sclerosis and psoriasis.

The subject of the present invention is a compound according to Formula (I)

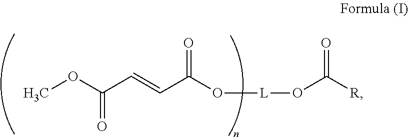

wherein

n is 1 or 2,

L is a an organic residue comprising 2 to 25 carbon atoms, and

—OOCR is a carboxylate of a nonsteroidal anti-inflammatory drug

or a pharmaceutically acceptable salt, hydrate, solvate, polymorph, and/or mixtures thereof.

It was found that the compounds of the present invention show unexpected properties, e.g. superior pharmaceutical and/or pharmacokinetic properties. In particular, the compounds show an appropriate anti-inflammatory effect such that advantageously lowered doses of the compound with regard to a NSAID can be applied to the patient, which reduces the negative adverse effects associated with NSAIDs.

Further, the present invention relates to a compound according to Formula (I) or a pharmaceutically acceptable salt, hydrate, solvate, polymorph, and/or mixtures thereof, which can be used as a medicament for the treatment and/or prevention of systemic diseases, autoimmune diseases, and/or inflammatory diseases, for example osteoarthritis, rheumatoid arthritis, multiple sclerosis and psoriasis.

Another subject is a pharmaceutical composition comprising the above-mentioned compound according to Formula (I).

### DETAILED DESCRIPTION OF THE INVENTION

In the context of this invention, the compound of the present invention is represented by the above Formula (I). Further, the compound may refer to pharmaceutically acceptable salts, hydrates, solvates, polymorphs and mixtures thereof. For example, the invention also refers to pharmaceutically acceptable salts of compounds according to Formula (I) or to solvates of salts or hydrates or polymorphs or the like. The same applies to all embodiments, e.g. to compounds of Formulae (II), (IIIa), (IIIb) and (IV) to (X) as shown below.

In a preferred embodiment n is 1 and L can be a substituted or unsubstituted divalent aliphatic residue with 2 to 25 carbon atoms or a substituted or unsubstituted aromatic residue with 5 to 12 carbon atoms.

In case that L is substituted, the one or more substituents can preferably be selected independently from one or more of the following substituents: halogen, nitro, nitrile, urea, phenyl, aldehyde, sulfate, amino, hydroxy, methoxy, mercapto, methylthio, phenyl, and ═O such that the corresponding —CO-group is formed.

Within the present application a divalent residue L can be regarded as a linking group being able to be bonded to two residues, wherein in the present embodiment (n=1) these residues are mono methyl fumarate (hereinafter referred to as MMF) and the carboxylate of a nonsteroidal anti-inflammatory drug.

An aliphatic residue is a non-aromatic hydrocarbon compound which can comprise, apart from carbons and hydrogen atoms, for example also oxygen, sulphur and nitrogen atoms.

An aromatic residue has a ring system which according to Hückel contains in conjugated double bonds, free electron pairs or unoccupied p-orbitals a number of 4n+2 (n=0, 1, 2, . . . ) delocalized electrons.

In a preferred embodiment L can be a substituted or unsubstituted alkylene group with 2 to 25 carbon atoms, a substituted or unsubstituted alkenylene group with 2 to 25 carbon atoms or a substituted or unsubstituted cyclic alkylene group with 3 to 12 carbon atoms.

An alkylene group with 2 to 25 carbon atoms can be a linear or a branched alkylene group with 2 to 25 carbon atoms, preferably a linear or branched alkylene group with 2 to 12 carbon atoms.

Alkylene groups with 2 to 25 carbon atoms can for example include ethylene, propylene, 2-methylpropylene, butylene, 2-methylbutylene, 3-methylbutylene, pentylene, sec.-pentylene, hexylene, ocytylene and dodecylene.

Cyclic alkylene with 3 to 12 carbon atoms can for example include cyclopropylene, cyclobutylene, cyclopentylene and cyclohexylene.

In a further preferred embodiment L can be a substituted or unsubstituted alkylene group with 3 to 25 carbon atoms, a substituted or unsubstituted alkenylene group with 3 to 25 carbon atoms or a substituted or unsubstituted cyclic alkylene group with 3 to 12 carbon atoms, wherein in the substituted or unsubstituted alkylene group with 3 to 25 carbon atoms, in the substituted or unsubstituted alkenylene group with 3 to 25 carbon atoms or in the substituted or unsubstituted cyclic alkylene group with 3 to 12 carbon atoms one or more —CH₂— group(s) can be substituted by an oxygen atom (—O—) to form an ether.

In a preferred embodiment L can be —((CH₂)₂O)ₘ—(CH₂)₂—, wherein m is 1 to 10, preferably 2 to 5. It is particularly preferred that m is 3 or 4, especially 3, such that the corresponding L is —((CH₂)₂O)₃—(CH₂)₂—. A particularly preferred compound according to Formula (I) can thus be represented by the following Formula (II)

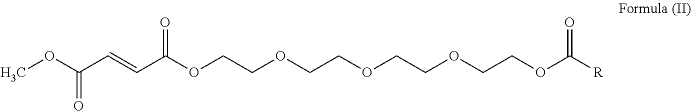

wherein OOCR is a carboxylate of nonsteroidal anti-inflammatory drug.

In an alternative embodiment L can be —(CH₂CHCH₃O)ₘ—(CH₂CHCH₃)— or —(CHCH₃CH₂O)ₘ—(CHCH₃CH₂)—, wherein m is 1 to 10, preferably 2 to 5. It is particularly preferred that m is 3 such that the corresponding L is —(CH₂CHCH₃O)₃—(CH₂CHCH₃)— or —(CHCH₃CH₂O)₃—(CHCH₃CH₂)—. A preferred compound according to Formula (I) can thus be represented by the following Formulae (IIIa) or (IIIb)

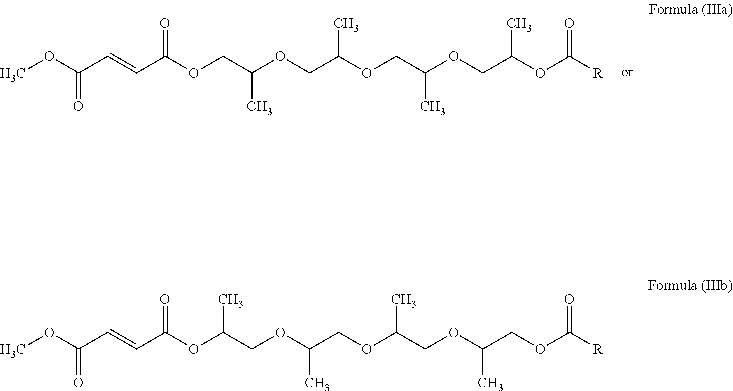

wherein —OOCR is a carboxylate of a nonsteroidal anti-inflammatory drug.

A compound according to Formula (I), wherein n=1, can preferably be synthesized via the following route:

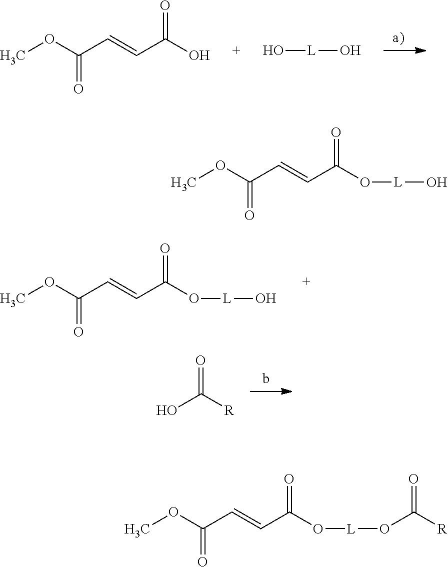

Preferably, in step a) HO-L-OH and MMF can be submitted to an esterification in an organic solvent in the presence of a coupling agent. A coupling agent is preferably a substance generally facilitating the formation of an ester or an amide. The coupling agent reacts with a carboxy group by forming a reactive intermediate which is subsequently further reacted with an alcohol or an amine to form the final product, i.e. an ester or an amide. Suitable coupling agents can be for example DCC (N,N′-dicyclohexylcarbodiimide), DIC (N,N′-diisopropylcarbodiimide), EDC×HCl (N-ethyl-N′-(3-methylaminopropyl)carbodiimide hydrochloride), CDI (carbonyldiimidazole), preferably EDC×HCl. It is further preferred that the coupling reaction is carried out in the presence of an auxiliary alkaline compound.

Suitable alkaline compounds are for example pyridine and amines, such as triethylamine, diisopropylethylamine and DMAP (4-(dimethylamino)pyridine), in particular DMAP.

A suitable organic solvent can for example be dichloromethane, chloroform, acetonitrile, dioxane, tetrahydrofuran and dimethylformamide.

Alternatively, MMF can be preferably reacted with thionyl chloride or oxalyl chloride, preferably oxalyl chloride, to form the corresponding acid chloride.

Subsequently, the corresponding acid chloride can be submitted to a reaction with HO-L-OH, preferably in an organic solvent such as dioxane, tetrahydrofuran, chloroform or dichloromethane. Further, the reaction of the acid chloride with HO-L-OH is preferably carried out in the presence of an auxiliary alkaline compound. Suitable alkaline compounds are for example pyridine and amines, such as triethylamine, DMAP (4-(dimethylamino)pyridine and diisopropylethylamine, preferably triethylamine.

Alternatively, the above acid chloride of MMF can be further transferred in activated esters like the para-nitrophenol ester.

Further alternatively, MMF can be reacted with acid chlorides, diphenylphosphoryl azide or chlorosulfonyl isocyanate to form (mixed) anhydrides. These mixed anhydrides can be also submitted to further reactions to obtain further forms of anhydrides. For example, the anhydride of monomethylfumarate can be obtained by said preparation.

Subsequently, an activated ester or MMF anhydride can be submitted to a reaction with HO-L-OH, preferably in an organic solvent such as dioxane, tetrahydrofuran, chloroform, acetone or dichloromethane. Further, the reaction of an activated ester or MMF anhydride with HO-L-OH is preferably carried out in the presence of an auxiliary alkaline compound. Suitable alkaline compounds are for example pyridine and amines, such as triethylamine, diisopropylethylamine and DMAP (4-(dimethylamino)pyridine), preferably DMAP.

Alternatively, the reaction of the activated ester or MMF anhydride with HO-L-OH can preferably be carried out in the absence of an auxiliary alkaline compound.

A suitable organic solvent can for example be dichloromethane, chloroform, acetonitrile, dioxane, tetrahydrofuran and dimethylformamide.

In a preferred embodiment one of the hydroxy groups of HO-L-OH can be protected with a protection group before being submitted to a reaction with MMF in the presence of a coupling agent or with the acid chloride of MMF or the anhydride of MMF. Such a protection group can for example be a trialkylsilyl group.

After the coupling reaction the protection can preferably be removed by a suitable reaction.

In step b) the product from step a) and HOOCR can be submitted to an esterification, preferably under the same conditions as described above with regard to step a).

In an alternatively preferred embodiment HO-L-OH and HOOCR can be submitted to an esterification in a first reaction step. In a second reaction step MMF and the product of said first step can be submitted to an esterification to achieve the compound according to Formula (I), wherein n=1. The conditions for esterification correspond to the ones described above.

In an alternative preferred embodiment in a compound according to Formula (I) n is 2 and L can be a substituted or unsubstituted trivalent aliphatic residue with 3 to 25 carbon atoms or a substituted or unsubstituted aromatic residue with 5 to 12 carbon atoms.

As far as the substituents, the aliphatic and aromatic residue are concerned the same applies as described above.

Further, the trivalent residue L can be regarded as a linking group being able to be bonded to three residues wherein in the present case these residues are two MMFs and one carboxylate of a nonsteroidal anti-inflammatory drug.

In a preferred embodiment L can be a substituted or unsubstituted alkylene group with 3 to 25 carbon atoms, a substituted or unsubstituted alkenylene group with 3 to 25 carbon atoms, or a substituted or unsubstituted cyclic alkylene group with 3 to 12 carbon atoms.

A substituted or unsubstituted alkylene group with 3 to 25 carbon atoms can be linear or branched.

Alkylene groups with 3 to 25 carbon atoms can for example include propylene, 2-methylpropylene, butylene, 2-methylbutylene, 3-methylbutylene, pentylene, sec.-pentylene, hexylene, ocytylene and dodecylene.

Cyclic alkylene with 3 to 12 carbon atoms can for example include cyclopropylene, cyclobutylene, cyclopentylene and cyclohexylene.

In a preferred embodiment L can be a substituted or unsubstituted alkylene group with 4 to 25 carbon atoms, a substituted or unsubstituted alkenylene group with 4 to 25 carbon atoms, or a substituted or unsubstituted cyclic alkylene group with 4 to 12 carbon atoms, wherein in the substituted or unsubstituted alkylene group with 4 to 25 carbon atoms, in the substituted or unsubstituted alkenylene group with 4 to 25 carbon atoms, or in the substituted or unsubstituted cyclic alkylene group with 4 to 12 carbon atoms one or more —CH₂— group(s) can be substituted by an oxygen atom (—O—) to form an ether.

In a particularly preferred embodiment L can be

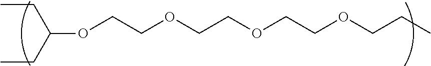

In an alternatively particularly preferred embodiment L can be

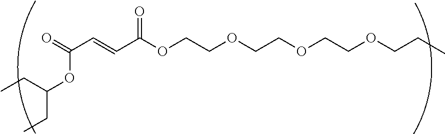

Thus, a particularly preferred compound according to Formula (I), wherein n=2, can be represented by the Formula (IV)

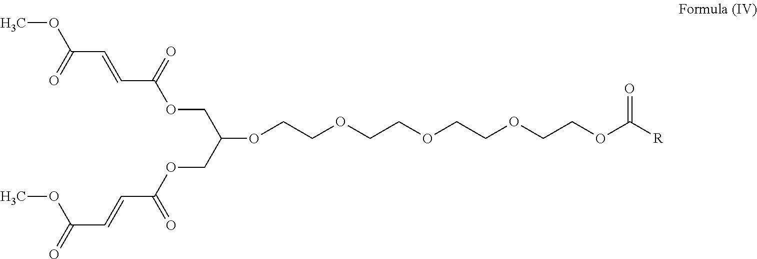

wherein OOCR is a carboxylate of a nonsteroidal anti-inflammatory drug.

Further, an alternatively particularly preferred compound according to Formula (I), wherein n=2, can be represented by the Formula (IV)

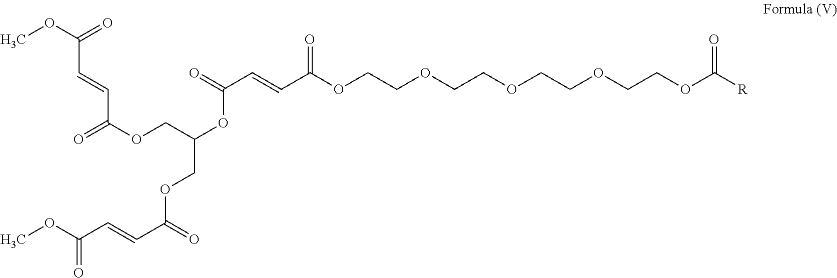

wherein OOCR is a carboxylate of a nonsteroidal anti-inflammatory drug.

A compound according to Formula (I), wherein n is 2, can preferably be synthesized via the following route:

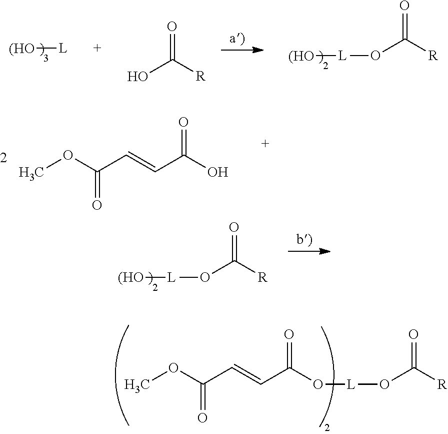

Preferably, in step a′) (HO)₃-L and HOOCR can be submitted to an esterification in an organic solvent. The conditions can preferably correspond to the conditions described above.

In a preferred embodiment one or two of the hydroxy groups of (HO)₃-L can be protected with a protection group before being submitted to a reaction with HOOCR. Such a protection group can for example be a trialkylsilyl or an acetal group.

After the coupling reaction the protection can preferably be removed by a suitable reaction.

In step b′) the product from step a′) and two equivalents of MMF can be submitted to an esterification, preferably under the same conditions as described above.

Alternatively preferred (HO)₃-L can be first submitted to a reaction with 2 equivalents of MMF and in a second step the resulting product is submitted to a reaction with HOOCR. Preferably the same conditions for esterification as above can be applied.

A compound according to Formula (I), wherein n is 2, can alternatively preferably be synthesized via the following route.

HOOCR can be reacted with a precursor of L. Suitable precursors of L can be for example diols, such as ethyleneglycol, diethyleneglycol, triethyleneglycol or tetraethyleneglycol. In a next step the resulting product can be activated. Activation can for example be a conversion of a hydroxy to a bromine group or alternatively the reaction of the hydroxy with a carboxylic acid anhydride, such as maleic anhydride, to the corresponding carboxylic acid. The activated compound can preferably be submitted to a further reaction to form the skeleton of L, for example by the reaction with 1,3-dichloropropane-2-ol. After a reaction with two equivalents of MMF a compound according to Formula (I), wherein n is 2, can be obtained.

Alternatively preferred, a compound according to Formula (I), wherein n is 2, can preferably be synthesized via one of the following routes:

Route A:

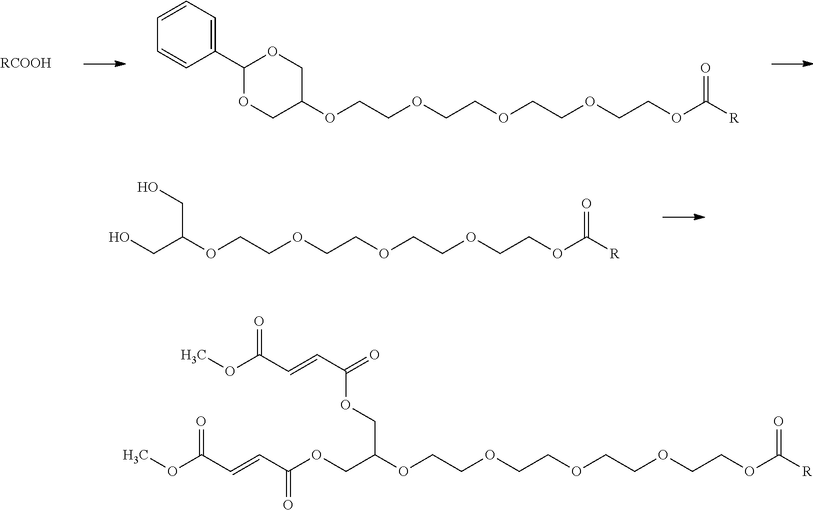

Route B:

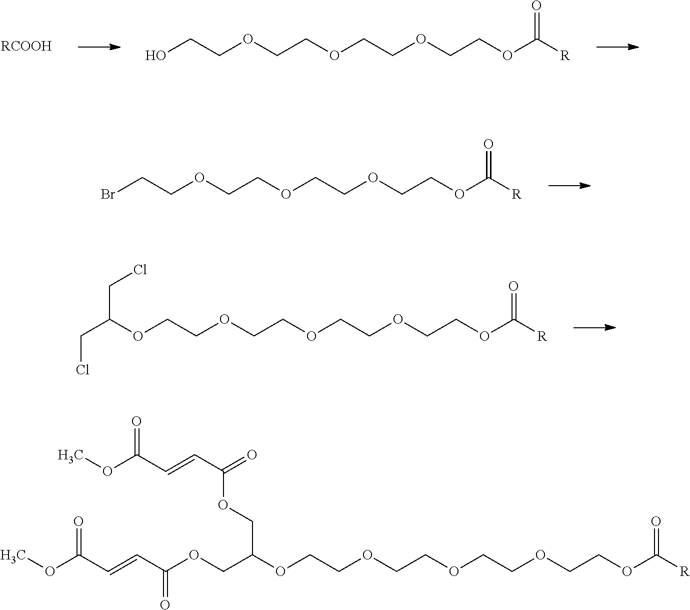

Route C:

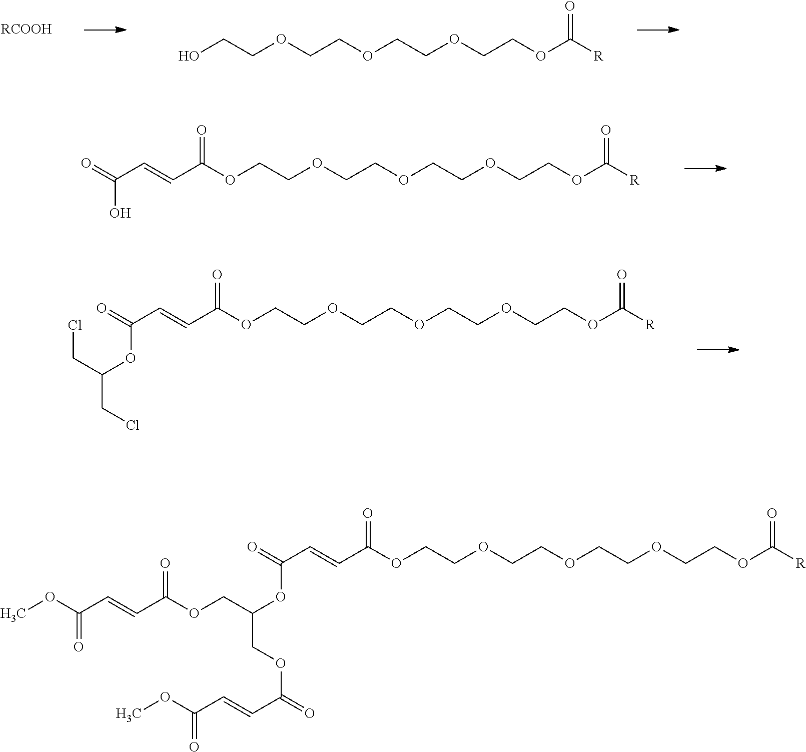

As described above the group L comprises several preferred embodiments. Also the group —OOCR, which is the carboxylate of a nonsteroidal anti-inflammatory drug, comprises several preferred embodiments.

Generally, nonsteroidal anti-inflammatory drugs are drugs which provide an analgesic, antipyretic and/or anti inflammatory effect. In the present case the nonsteroidal anti-inflammatory drugs shall comprise a carboxylic acid group, which bonds to the linker L.

In a preferred embodiment of the invention —OOCR is the carboxylate of 2-(2,3-dimethylphenyl)aminobenzoic acid (mefenamic acid), 2-{[3-(trifluoromethyl)phenyl]amino}benzoic acid (flufenamic acid), 2-[(2,6-dichloro-3-methylphenyl)amino]benzoic acid (meclofenamic acid), 2-{[3-(trifluoromethyl)phenyl]amino}nicotinic acid (niflumic acid), 2-[2-[2-[(2,6-dichlorophenyl)amino]phenyl]acetyl]oxyacetic acid (aceclofenac), 2-[2-[1-(4-chlorobenzoyl)-5-methoxy-2-methylindol-3-yl]acetyl]oxyacetic acid (acemetacin), 2-(2-(2,6-dichlorophenylamino)phenyl)acetic acid (diclofenac), 2-[(2-chloro-6-fluorophenyl)amino]-5-methylphenyl}acetic acid (lumiracoxib), 2-(1,8-Diethyl-4,9-dihydro-3H-pyrano[3,4-b]indol-1-yl)acetic acid (etodolac), 2-{1-[(4-chlorophenyl)carbonyl]-5-methoxy-2-methyl-1H-indol-3-yl}acetic acid (indometacin), 5-Benzoyl-2,3-dihydro-1H-pyrrolizine-1-carboxylic acid (ketorolac), 2-[2-amino-3-(4-bromobenzoyl)phenyl]acetic acid (bromfenac), (RS)-2-(4-(2-methylpropyl)phenyl)propanoic acid (ibuprofen), (2S)-2-[4-(2-methylpropyl)phenyl]propanoic acid (dexibuprofen), (+)-(S)-2-(6-methoxynaphthalen-2-yl)propanoic acid (naproxen), (RS)-2-(3-benzoylphenyl)propanoic acid (ketoprofen), (2S)-2-[3-(benzoyl)phenyl]propanoic acid (desketoprofen), (RS)-2-(2-fluorobiphenyl-4-yl)propanoic acid (flurbiprofen), 2-[2-(4-chlorophenyl)-1,3-benzoxazol-5-yl]propanoic acid (benoxaprofen), (RS)-2-(5-benzoyl-2-thienyl)propanoic acid (tiaprofenic acid), or 2-(acetoxy)benzoic acid (aspirin) or [6-(4-chlorophenyl)-2,2-dimethyl-7-phenyl-2,3-dihydro-1H-pyrrolizin-5-yl]acetic acid (licofelone), preferably the carboxylate of (RS)-2-(4-(2-methylpropyl)phenyl)propanoic acid (ibuprofen), (2S)-2-[4-(2-methylpropyl)phenyl]propanoic acid (dexibuprofen), (+)-(S)-2-(6-methoxynaphthalen-2-yl)propanoic acid (naproxen), (RS)-2-(2-fluorobiphenyl-4-yl)propanoic acid (flurbiprofen) or [6-(4-chlorophenyl)-2,2-dimethyl-7-phenyl-2,3-dihydro-1H-pyrrolizin-5-yl]acetic acid (licofelone), in particular the carboxylate of [6-(4-chlorophenyl)-2,2-dimethyl-7-phenyl-2,3-dihydro-1H-pyrrolizin-5-yl]acetic acid (licofelone).

In a preferred embodiment —OOCR is not the carboxylate of 2-(acetoxy)benzoic acid (aspirin).

Particularly preferred examples of compounds according to Formula (I) are shown below in Formula (VI)-(X).

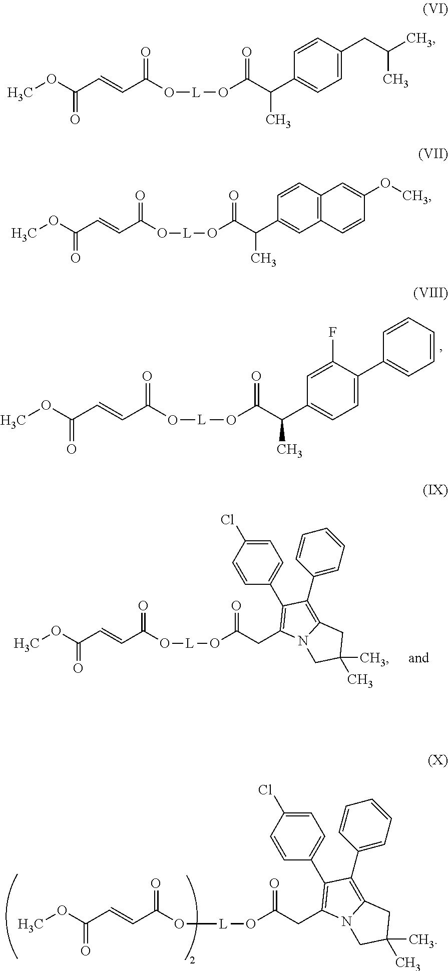

Preferably, for formulae (VI)-(X) the above illustrations for the linking group L, where n=1 apply. In a preferred embodiment L can be —((CH₂)₂O)ₘ—(CH₂)₂—, wherein m is 1 to 10, preferably 2 to 5. It is particularly preferred that m is 3 such that the corresponding L is —((CH₂)₂O)₃—(CH₂)₂—.

Further, the present invention relates to a compound according to Formula (I) wherein n is 1 or 2, L is an organic residue comprising 2 to 25 carbon atoms, and —OOCR is a carboxylate of a nonsteroidal anti-inflammatory drug, or a pharmaceutically acceptable salt, hydrate, solvate, polymorph, co-crystal and/or mixtures thereof for use as a medicament.

A further subject of the invention is a compound according to Formula (I), wherein n is 1 or 2, L is an organic residue comprising 2 to 25 carbon atoms and —OOCR is a carboxylate of a nonsteroidal anti-inflammatory drug or a pharmaceutically acceptable salt, hydrate, solvate, polymorph and/or mixtures thereof for use in the treatment and/or prevention of systemic diseases, autoimmune diseases or inflammatory diseases.

To an above compound according to Formula (I) for use in the treatment and/or prevention of systemic diseases, autoimmune diseases or inflammatory diseases the same applies as to a compound according to Formula (I) for use as a medicament.

Systemic diseases do not just affect single organs. Instead, these diseases are known to affect a number of organs and tissues or even the body as a whole.

People having an autoimmune disease usually suffer from their immune system mistakenly attacking the cells of their own organism and thus incorrectly responding to substances normally present in the body.

An inflammation can be defined as the response of the body to the occurrence of harmful stimuli which can result in pain, heat, redness, swelling and loss of function of the affected organ.

It is possible that some of the above-mentioned diseases cannot be allocated in one single group of the above-mentioned groups, since they show the symptoms of more than one of them.

In a preferred embodiment the compounds of the present invention can be used in the treatment of rheumatoid arthritis, osteoarthritis, psoriatic arthritis, reactive arthritis, migraine, (acute) gout, metastatic bone pain, muscle stiffness and pain due to Parkinson's disease, ileus and renal colic.

In a further aspect of the present invention it has been unexpectedly found, that the above-mentioned compounds according to Formula (I) can be advantageously used in the treatment of multiple sclerosis, rheumatoid arthritis and psoriasis, preferably multiple sclerosis. Said compounds can e.g. be used in the treatment of the following types of multiple sclerosis: relapsing-remitting, primary-progressive, secondary-progressive and progressive-relapsing. In a preferred embodiment the compounds of the present invention are used in the treatment of relapsing-remitting multiple sclerosis.

In a further aspect of the invention, in the compound according Formula (I), R can be regarded as a substance which is hydrolyzed—for example by esterases in the intestine—to monomethyl fumarate (MMF). Such a compound can be regarded as a prodrug of MMF. MMF is reported to be a metabolite of dimethyl fumarate (DMF) and can be characterized by the following chemical Formula (A):

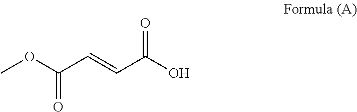

It is indicated that, in vivo, DMF and MMF show about the same efficacy, in particular on the transcription factor Nrf2. However, compared to DMF, the compound according Formula (I) shows advantageous pharmacokinetic properties. In particular, the present compounds are hydrolysed differently to MMF, for example more rapidly or more slowly, especially more slowly than DMF in the human body (or under respective in-vitro conditions) and cause fewer undesirable side effects than associated with DMF.

Further, the present invention also provides a pharmaceutical composition comprising the compound according to the present invention, i.e. a pharmaceutical composition comprising a compound according to Formula (I), for use as a medicament and optionally pharmaceutical excipients.

In a preferred embodiment the pharmaceutical composition comprises
(i) 0.01 to 10 mmol, more preferably 0.05 to 7.5 mmol, still more preferably 0.25 to 6 mmol and particularly preferred 0.35 to 5 mmol of a compound according to Formula (I) for use as a medicament
(ii) pharmaceutical excipient(s).

When —OOCR is the carboxylate of (RS)-2-(4-(2-methylpropyl)phenyl)propanoic acid (ibuprofen) the pharmaceutical composition can preferably comprise 1 to 7 mmol, more preferably 2 to 6 mmol, in particular 3 to 5 mmol, especially 3.87 mmol of the corresponding compound according to Formula (I) and pharmaceutical excipients.

When the —OOCR is the carboxylate of (+)-(S)-2-(6-methoxynaphthalen-2-yl)propanoic acid (naproxen), the pharmaceutical composition can preferably comprise 0.75 to 5 mmol, more preferably 1 to 4 mmol, in particular 1.5 to 3 mmol, especially 2.17 mmol of the corresponding compound according to Formula (I) and pharmaceutical excipients.

When —OOCR is the carboxylate of [6-(4-chlorophenyl)-2,2-dimethyl-7-phenyl-2,3-dihydro-1H-pyrrolizin-5-yl]acetic acid (licofelone), the pharmaceutical composition can preferably comprise 0.25 to 4 mmol, more preferably 0.3 to 2.5 mmol, in particular 0.5 to 1.5 mmol, especially 1.05 mmol of the corresponding compound according to Formula (I) and pharmaceutical excipients.

When —OOCR is the carboxylate of (RS)-2-(2-fluorobiphenyl-4-yl)propanoic acid (flurbiprofen), the pharmaceutical composition can preferably comprise 0.05 to 1.5 mmol, more preferably 0.1 to 1.25 mmol, in particular 0.19 to 1 mmol, especially 0.385 mmol of the corresponding compound according to Formula (I) and pharmaceutical excipients.

The pharmaceutical formulation can preferably be further processed to or be in a form suitable for oral administration, preferably in form of a solid oral doasage form. Preferably the pharmaceutical composition is in form of a tablet or a capsule, in particular in form of a tablet.

The pharmaceutical composition and/or the oral dosage form of the present invention can be prepared by methods well known to a person skilled in the art such as dry or wet granulation or direct compression.

The pharmaceutical composition can additionally contain one or more pharmaceutically acceptable excipient(s), such as fillers, binders, glidants, disintegrants and lubricants. Suitable excipients are for example disclosed in “Lexikon der Hilfsstoffe für Pharmazie, Kosmetik and angrenzende Gebiete”, published by H. P. Fielder, 4^{th} Edition and “Handbook of Pharmaceutical Excipients”, 3^{rd} Edition, published by A. H. Kibbe, American Pharmaceutical Association, Washington, USA, and Pharmaceutical Press, London.

The term filler generally means substances which serve to form the body of the tablet in the case of tablets with small amounts of active agent (e.g. less than 60% by weight). This means that fillers “dilute” the active agent(s) in order to produce an adequate tablet compression mixture. The normal purpose of fillers therefore is to obtain a suitable tablet size. Fillers may fulfil several requirements such as being chemically inert, non-hygroscopic, biocompatible, easy processable and possessing good biopharmaceutical properties. Fillers can be present in an amount of 0 to 80% by weight, preferably in an amount of 10 to 60% by weight based on the total weight of the composition.

A binder is generally a substance which is capable of increasing the strength of the resulting dosage form, especially the resulting tablets. Binders can be present in an amount of 0 to 30% by weight, preferably in an amount of 2 to 15% by weight based on the total weight of the composition.

Glidants can be used to improve the flowability. The glidant can be present for example in an amount of 0 to 3% by weight, preferably in an amount of 0.2 to 2% by weight based on the total weight of the composition.

Disintegrants are compounds which enhance the ability of the dosage form, preferably the ability of the tablet, to break into smaller fragments when in contact with a liquid, preferably water. The disintegrant can be present in an amount of 0 to 20% by weight, preferably in an amount of 1 to 15% by weight based on the total weight of the composition.

Lubricants are generally used in order to reduce sliding friction. In particular, the intention is to reduce the sliding friction found during tablet pressing between the punch moving up and down in the die and the die wall, on the one hand, and between the edge of the tablet and the die wall, on the other hand. Preferably, lubricants can be present in an amount of 0 to up to 5 wt %, more preferably of 0.1 to 4 wt % based on the total weight of the dosage form.

In a preferred embodiment the excipients are chosen such that the resulting formulation is a gastric juice-resistant formulation. In a preferred embodiment the formulation of the present invention does not show significant drug release under acidic conditions. In particular, the in-vitro drug release after 2 hours is less than 10%, preferably 0 to 9.9%, more preferably 0 to 5%, still more preferably 0.001 to 3%, measured according to USP, Apparatus II, paddle, 0.1 N HCl, 37° C., 50 rpm.

It is further preferred that the pharmaceutical composition is processed into an oral dosage form. The oral dosage form, preferably a tablet or a capsule, more preferably a tablet, can preferably be coated, preferably be film coated.

In the present invention, the following three types of film coatings are possible:
film coating without affecting the release of the active ingredient,
gastric juice-resistant film coatings,
retard film coatings.

In a preferred embodiment a film coating without affecting the release of the active agent can be used.

It is alternatively preferred that the present tablet is coated with a gastric juice-resistant film coating. Alternatively, a capsule comprising a gastric juice-resistant film coating can be used.

The gastric juice-resistant film coating preferably is a film coating being stable in the pH range of about 0.7 to 3.0, but in an environment with a pH value of 5 to 9 the gastric juice-resistant film coating preferably dissolves and the drug can be released.

The coating is preferably free of active ingredient. It is further preferred that the thickness of the coating is usually 1 μm to 2 mm, preferably from 5 to 100 μm.

In a preferred embodiment the pharmaceutical composition can be administered one to three times a day, preferably once or twice a day, more preferably once a day.

Further, the present invention relates to a method for treating and/or preventing inflammatory diseases, preferably rheumatoid arthritis, osteoarthritis, psoriatic arthritis, reactive arthritis, migraine, (acute) gout, metastatic bone pain, muscle stiffness and pain due to Parkinson's disease, ileus and renal colic, comprising administering to a subject in need thereof a therapeutically effective amount of the compound of the invention, in particular a compound according to Formula (I) wherein the residues are defined as above, or the pharmaceutical composition of the invention. To this compound and this pharmaceutical composition the same explanation (e.g. regarding combination of possible embodiments) apply as to the compound and the pharmaceutical composition as described above, respectively.

Further, the present invention relates to a method for treating and/or preventing systemic diseases, autoimmune diseases and/or inflammatory diseases, preferably multiple sclerosis, rheumatoid arthritis or psoriasis, comprising administering to a subject in need thereof a therapeutically effective amount of the compound of the invention, in particular a compound according to Formula (I) where the residues are defined as above, or the pharmaceutical composition of the invention. To this compound and this pharmaceutical composition the same explanation (e.g. regarding combination of possible embodiments) apply as to the compound and the pharmaceutical composition as described above, respectively. In an embodiment the treatment of multiple sclerosis is especially preferred.

The invention is illustrated by the following examples.

### EXAMPLES

### Example 1: (E)-But-2-enedioic acid 2-(2-hydroxy-ethoxy)-ethyl ester methyl ester

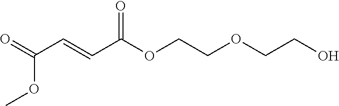

16.31 g (0.15 mol) diethylenglycol (DEG), 7.07 g (36.9 mmol) N-ethyl-N′-(3-dimethyl-aminopropyl)carbodiimide hydrochloride (EDC×HCl), and 0.19 g (1.5 mmol) 4-(dimethylamino)pyridine (DMAP) were dissolved in 40 ml tetrahydrofuran (THF). 4 g (30.8 mmol) monomethylfumarate dissolved in 66 ml THF were dropped into the DEG-solution at room temperature (RT) within 35 minutes. The reaction mixture was kept under continuous stirring at RT for 1.5 h. Stirring was stopped and a biphasic system was obtained; the lower layer was discarded and the upper layer was evaporated. The obtained crude product (colorless oil) was subjected to flash chromatography (100% ethyl acetate) twice. The product was dried under high vacuum at RT for 5 hours to yield the product as colorless oil (2.7 g; 12.3 mmol).

¹H NMR (400 MHz, CDCl₃) δ [ppm]: 2.44-2.49 (s, 1H) 3.53-3.59 (m, 2H) 3.69 (s, 4H) 3.75 (s, 3H) 4.31 (m, J=4.70, 4.70 Hz, 2H) 6.82 (s, 2H)

### Example 2: (E)-But-2-enedioic acid 2-{2-[2-(2-hydroxy-ethoxy)-ethoxy]-ethoxy}-ethyl ester methyl ester

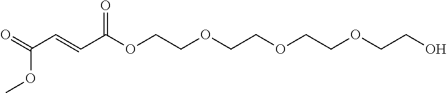 [SMILES: COC(=O)/C=C/C(=O)OCCOCCOCCOCCO]

13.4 g (69.2 mmol) tetraethylenglycol (TEG), 5.3 g (27.7 mmol) EDC×HCl, 0.14 g (1.2 mmol) DMAP were dissolved in 50 ml THF. 3 g (23 mmol) monomethyl fumarate, dissolved in 50 ml THF, were added into the TEG-solution at RT. The reaction mixture was kept under continuous stirring at RT for 3 h. Stirring was stopped and a biphasic layer was obtained, the lower layer was discarded and the upper layer evaporated. The obtained crude product was subjected to flash chromatography (100% ethyl acetate) twice. The product was dried under high vacuum at RT for 5 hours to yield the product as colorless oil (3.14 g; 10.3 mmol)

¹H NMR (400 MHz, CDCl₃) δ [ppm]: 2.53 (s, 1H) 3.55-3.60 (m, 2H) 3.64 (s, 8H) 3.67-3.74 (m, 4H) 3.78 (s, 3H) 4.32-4.35 (m, 2H) 6.86 (s, 2H)

### Example 3: ((E)-But-2-enedioic acid 2-(2-{2-[2-(4-chloro-phenyl)-6,6-dimethyl-1-phenyl-6,7-dihydro-5H-pyrrolizin-3-yl]-acetoxy}-ethoxy)-ethyl ester methyl ester

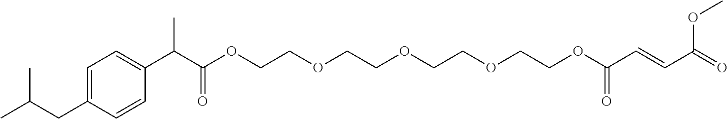 [SMILES: COC(=O)/C=C/C(=O)OCCOCCOCCOCCOC(=O)C(C)c1ccc(CC(C)C)cc1]

0.27 g (1.3 mmol) 2-(4-Isobutyl-phenyl)-propionic acid (Ibuprofen) was dissolved in 10 ml THF. To this solution, 0.33 g EDC×HCl and 0.01 g DMAP were added. A solution of 0.48 g (1.6 mmol) (E)-But-2-enedioic acid 2-{2-[2-(2-hydroxy-ethoxy)-ethoxy]-ethoxy}-ethyl ester methyl ester in 5 ml THF was dropped into the free acid solution over a period of 10 minutes. The reaction mixture was stirred overnight. A clear solution was formed with syrupy white precipitate, the THF layer was decanted off and was evaporated. The oily product was subjected to flash chromatography (ethyl acetate/heptane 2:1 (v/v) to yield the product as colorless oil (0.42 g; 0.8 mmol).

¹H NMR (400 MHz, DMSO-d₆) δ [ppm]: 0.82 (s, 3H) 0.84 (s, 3H) 1.35 (d, J=7.04 Hz, 3H) 1.72-1.85 (m, 1H) 2.39 (d, J=7.43 Hz, 2H) 3.30 (s, 1H) 3.40-3.44 (m, 4H) 3.45-3.55 (m, 6H) 3.62-3.66 (m, 2H) 3.72 (s, 4H) 4.05-4.16 (m, 2H) 4.25 (m, J=4.70 Hz, 2H) 6.76 (s, 2H) 7.05-7.10 (m, 2H) 7.14-7.19 (m, 2H)

LC-MS: tᵣ: 5.0 min.; m/z: 495 [M+H]⁺ (method E)

### Example 4: ((E)-But-2-enedioic acid 2-[2-(2-{2-[(S)-2-(6-methoxy-naphthalen-2-yl)-propionyloxy]-ethoxy}-ethoxy)-ethoxy]-ethyl ester methyl ester

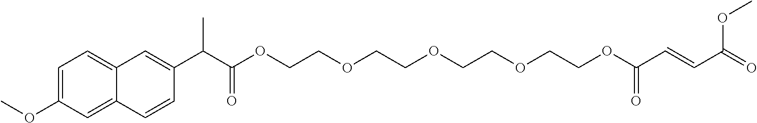

0.23 g (1 mmol) (+)-(S)-2-(6-methoxynaphthalen-2-yl)propanoic acid (Naproxen) and 0.34 g (1.1 mmol) (E)-But-2-enedioic acid 2-{2-[2-(2-hydroxy-ethoxy)-ethoxy]-ethoxy}-ethyl ester methyl ester were dissolved in 5 ml THF. To this solution 0.23 g (1.2 mmol) EDC×HCl and 10 mg DMAP were added. The reaction mixture was stirred overnight (0/N). A clear solution was obtained with syrupy white precipitate The THF layer was decanted off and the solvent was evaporated. The oily product was subjected to flash chromatography (80 g silica, ethylacetate/n-heptane 2:1 (v/v)) to yield the product as colorless oil (0.35 g; 0.7 mmol).

¹H NMR (400 MHz, DMSO-d₆) δ [ppm]: 1.45 (d, J=7.04 Hz, 3H) 3.38 (d, J=4.30 Hz, 6H) 3.45-3.49 (m, 2H) 3.50-3.55 (m, 2H) 3.59-3.64 (m, 2H) 3.72 (s, 3H) 3.84 (s, 3H) 3.90 (q, J=7.04 Hz, 1H) 4.12 (q, J=4.30 Hz, 2H) 4.21-4.27 (m, 2H) 6.75 (s, 2H) 7.13 (dd, J=8.99, 2.74 Hz, 1H) 7.27 (d, J=2.35 Hz, 1H) 7.38 (dd, J=8.41, 1.76 Hz, 1H) 7.70 (s, 1H) 7.76 (t, J=8.41 Hz, 2H)

LC-MS: tᵣ: 6.3 min.; m/z: 541 [M+H]⁺ (method A)

### Example 5: O4-[2-[2-[2-[2-[(2R)-2-(3-fluoro-4-phenyl-phenyl)propanoyl-]oxyethoxy]-ethoxy]ethoxy]ethyl]O1-methyl-(E)-but-2-enedioate

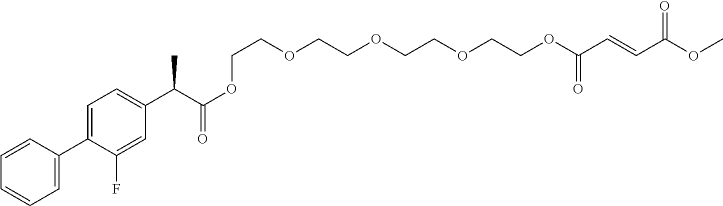 [SMILES: COC(=O)/C=C/C(=O)OCCOCCOCCOCCOC(=O)[C@H](C)c1ccc(-c2ccccc2)c(F)c1]

0.5 g (2.0 mmol) R-2-(2-fluorobiphenyl-4-yl)propanoic acid ((R)-flurbiprofen), 0.47 g (2.5 mmol) EDC×HCl, 10 mg DMAP and 0.69 g (2.3 mmol) (E)-But-2-enedioic acid 2-{2-[2-(2-hydroxy-ethoxy)-ethoxy]-ethoxy}-ethyl ester methyl ester (2) were dissolved in 10 ml THF. The reaction mixture was stirred 0/N at RT. The solvent was evaporated at 50° C. The obtained raw product was dissolved in ethyl acetate and water. After filtration, the organic layer was separated, dried over sodium sulfate and the solvent was evaporated. The crude product was subjected to flash chromatography (80 g silica, ethylacetate/n-heptane 2:1 (v/v)) to yield the product as slightly yellow oil.

1H-NMR (400 MHz, DMSO-d6) δ[ppm]: 1.42 (d, J=7.04 Hz, 3H) 3.45 (s, 4H) 3.45-3.52 (m, 4H) 3.53-3.58 (m, 2H) 3.59-3.64 (m, 2H) 3.72 (s, 3H) 3.89 (q, J=7.04 Hz, 1H) 4.15 (dd, J=5.47, 3.52 Hz, 2H) 4.24 (dd, J=5.47, 3.91 Hz, 2H) 6.74 (s, 2H) 7.20-7.25 (m, 2H) 7.35-7.41 (m, 1H) 7.46 (td, J=7.92, 4.89 Hz, 3H) 7.49-7.55 (m, 2H).

LC-MS: tᵣ: 3.7 min.; m/z: 550 [M+NH₄]⁺ and m/z 555 [M+Na]⁺ (method A)

### Example 6: O4-[2-[2-[2-(4-chlorophenyl)-6,6-dimethyl-1-phenyl-5,7-dihydropyrrolizin-3-yl]acetyl]oxyethyl] O1-methyl (E)-but-2-enedioate

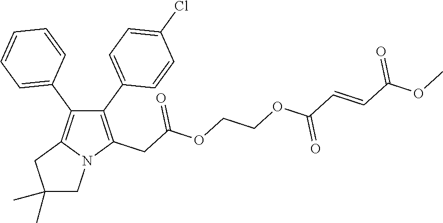 [SMILES: COC(=O)/C=C/C(=O)OCCOC(=O)Cc1c(-c2ccc(Cl)cc2)c(-c2ccccc2)c2n1CC(C)(C)C2]

1.5 g (3.5 mmol) 2-hydroxyethyl 2-[2-(4-chlorophenyl)-6,6-dimethyl-1-phenyl-5,7-dihydropyrrolizin-3-yl]acetate, 1 g (5.2 mmol) EDC×HCl, 20 mg (0.2 mmol) DMAP and 0.55 g (4.2 mmol) monomethylfumarate were dissolved in 20 ml THF. The reaction mixture was stirred 0/N at RT. Stirring was stopped (solution with a syrupy brown precipitate) and the solvent was evaporated yielding a brown syrup. After addition of 100 ml water the mixture was extracted with 3×100 ml ethyl acetate. The solvent was evaporated and the crude product subjected to flash chromatography (80 g silica gel, ethyl acetate/n-heptane 50:50 (v/v), flow 30 mL/min.) to yield the product as yellow oil, which was dried at 4×10−2 mbar at RT for 1 hour. To the oily product, 30 ml n-pentane was added and the mixture was stirred 0/N at RT which resulted in the formation of a precipitate. The solid was filtered off and dried under vacuum at 30° C. for 30 minutes.

¹H-NMR (400 MHz, DMSO-d₆) δ [ppm]: 1.20 (s, 6H) 2.76 (s, 2H) 3.55 (s, 2H) 3.70 (s, 5H) 4.30-4.39 (m, 4H) 6.73 (s, 2H) 6.94 (d, J=7.43 Hz, 2H) 7.00-7.07 (m, 3H) 7.12-7.18 (m, 2H) 7.29 (d, J=7.82 Hz, 2H).

LC-MS (ESI⁺): m/z 536 [M+H]⁺

### Example 7: (E)-But-2-enedioic acid 2-(2-{2-[2-(4-chloro-phenyl)-6,6-dimethyl-1-phenyl-6,7-dihydro-5H-pyrrolizin-3-yl]-acetoxy}-ethoxy)-ethyl ester methyl ester

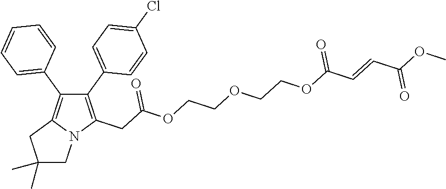 [SMILES: COC(=O)/C=C/C(=O)OCCOCCOC(=O)Cc1c(-c2ccc(Cl)cc2)c(-c2ccccc2)c2n1CC(C)(C)C2]

1 g (2.6 mmol) 6-(4-chlorophenyl)-2,2-dimethyl-7-phenyl-2,3-dihydro-1H-pyrrolizin-5-yl]acetic acid (Licofelone), 0.6 g (3.2 mmol) EDC×HCl, 0.02 g (0.1 mmol) 4-(dimethylamino)pyridine (DMAP) and 0.632 g (2.9 mmol) (E)-But-2-enedioic acid 2-(2-hydroxy-ethoxy)-ethyl ester methyl ester (were dissolved in THF (10 ml). The reaction mixture was kept under continuous stirring at room temperature for ˜1.5 h. Stirring was stopped (bright yellow solution with a syrupy white precipitate) and the solvent was evaporated yielding a bright yellow syrup. Water (50 ml) was added and the aqueous layer was extracted with ethylacetate 3 times (3×100 ml). The solvent was evaporated and the crude product subjected to flash chromatography (dichloromethane/MeCN 4:1) to yield the product as yellow oil, which was dried at 7×10−2 mbar at rt for 5 hours to afford the product as yellow syrupy product (1.01 g; 1.7 mmol).

¹H NMR (400 MHz, acetone-d₆) δ [ppm]: 1.28-1.32 (m, 6H) 2.79-2.83 (m, 2H) 3.58 (s, 2H) 3.72-3.80 (m, 7H) 3.82 (s, 2H) 4.24-4.28 (m, 2H) 4.32-4.35 (m, 2H) 6.79 (s, 2H) 7.03-7.07 (m, 3H) 7.14-7.19 (m, 4H) 7.30 (d, J=8.21 Hz, 2H)

LC-MS: tᵣ: 13.5 min.; m/z: 580 [M+H]⁺ (method B)

### Example 8: (E)-But-2-enedioic acid 2-{2-[2-(2-{2-[2-(4-chloro-phenyl)-6,6-dimethyl-1-phenyl-6,7-dihydro-5H-pyrrolizin-3-yl]-acetoxy}-ethoxy)-ethoxy]-ethoxy}-ethyl ester methyl ester

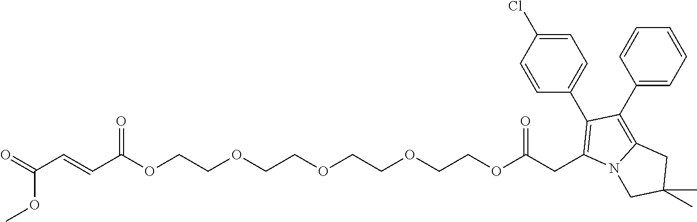 [SMILES: COC(=O)/C=C/C(=O)OCCOCCOCCOCCOC(=O)Cc1c(-c2ccc(Cl)cc2)c(-c2ccccc2)c2n1CC(C)(C)C2]

1 g (2.6 mmol) 6-(4-chlorophenyl)-2,2-dimethyl-7-phenyl-2,3-dihydro-1H-pyrrolizin-5-yl]acetic acid (Licofelone), 0.6 g (3.2 mmol) EDC×HCl, 20 mg (0.1 mmol) DMAP and 0.89 g (2.9 mmol) (E)-But-2-enedioic acid 2-{2-[2-(2-hydroxy-ethoxy)-ethoxy]-ethoxy}-ethyl ester methyl ester were dissolved in 30 ml THF. During 0/N stirring at RT, a bright yellow solution with a syrupy white precipitate was formed. The solvent was evaporated, to the bright yellow syrup 50 ml water were added and the aqueous layer was extracted with 3×100 ml ethyl acetate. The organic layers were combined, solvent was evaporated and the crude product subjected to flash chromatography (ethyl acetate/n-heptane 50:50 (v/v)) to yield the product as yellow oil, which was dried at 17 mbar at room temperature for 5 hours to afford the product as yellow syrupy product (1.1 g; 1.6 mmol).

¹H NMR (400 MHz, CDCl₃) δ [ppm]: 1.29 (s, 6H) 2.16 (s, 1H) 2.84 (s, 2H) 3.55 (s, 2H) 3.63 (d, J=2.35 Hz, 8H) 3.68-3.76 (m, 6H) 3.80 (s, 3H) 4.25-4.30 (m, 2H) 4.32-4.36 (m, 2H) 6.88 (s, 2H) 7.02-7.09 (m, 3H) 7.11-7.15 (m, 2H) 7.18 (s, 2H) 7.21-7.27 (m, 2H)

LC-MS: tᵣ: 12.3 min.; m/z: 688 [M+H]⁺ (method C)

Analytical Methods:

Nuclear Magnetic Resonance Spectroscopy:

Instrument: Varian Mercury 400 Plus NMR Spectrometer, Oxford AS, 400 MHz.

HPLC/UV/(Ion-Trap) MS:

Instrument: Agilent, HP 1200

Column: Phenomenex Kinetex C18, 150*4.6 mm, 2.6 μm

Oven temperature: 40° C.

Injection volume: 3 μl

Flow: 0.8 ml/min (method A, E, F); 1.0 ml/min (methods B, C and D)

coupled with

Instrument: Esquire HCT (Bruker Daltonics)

MS parameters:

Dry temperature: 320° C.

Nebulizer: 65.0 psi

Dry gas: 8.0 l/min

Ionization (polarity): electrospray (positive)

Scan range: m/z 50-1000

HPLC Methods:

Method A

Solvent A: acetonitrile

Solvent B: 0.2% formic acid, 0.1% HFBA (heptafluorobutyric acid)

| time [min] | solvent B [%] |
|---|---|
| 0.00 | 65 |
| 10.00 | 15 |
| 12.00 | 15 |
| 12.50 | 65 |
| 17.50 | 65 |

Method B

Solvent A: acetonitrile

Solvent B: 0.1% formic acid, HFBA pH=4

| time [min] | solvent B [%] |
|---|---|
| 0.00 | 80 |
| 2.00 | 80 |
| 10.00 | 30 |
| 11.00 | 15 |
| 15.00 | 15 |
| 15.10 | 80 |
| 22.00 | 80 |

Method C

Solvent A: acetonitrile; 0.2% formic acid; 0.1% HFBA

Solvent B: 0.2% formic acid, 0.1% HFBA

| time [min] | solvent B [%] |
|---|---|
| 0.00 | 60 |
| 15.00 | 5 |
| 16.00 | 5 |
| 16.10 | 60 |
| 22.00 | 60 |

Method E

Solvent A: acetonitrile

Solvent B: 0.2% formic acid, 0.1% HFBA

| time [min] | solvent B [%] |
|---|---|
| 0.00 | 30 |
| 8.00 | 15 |
| 12.00 | 15 |
| 12.10 | 30 |
| 18.00 | 30 |

### Example 9: Metabolic Stability of NSAID in Minipig Intestinal Fluid—Quantification of NSAID (Licofelone)

a) Preparation and Storage of Minipig Intestinal Fluid (IF)

Intestinal tissue/fluid and enterocyte samples were taken from a 10 month old, female Gottingen SPF minipig. The body weight was 21 kg. The minipig was fasted for approximately 28 hours before sampling of intestinal fluid/tissues and enterocytes. On the day of sampling, the minipig was weighed and anaesthetised. The animal was killed by exsanguination before sampling of intestinal fluid. An intestinal segment was ligated at both ends before removal. The isolated tissue was placed in isotonic saline and opened by a longitudinal cut for sampling of intestinal fluid.

The intestinal tissue from each segment was transferred into a Centrifuge Tube, immersed in 10 ml 50 mM phosphate buffer, pH 6.8 and frozen at −70° C.

b) Incubation Experiments with Minipig Intestinal Fluid (without FaSSIF)

In a HPLC glass vial, 8 μl of stock solution were mixed with 792 μl diluted IF (1 vol IF+4 vol 2% bovine serum albumin in FaSSIF) and the mixture was stirred (250 rpm) in a water bath (T=37° C.).

Immediately after mixing as well as at t=30 min, 60 min, 120 min and 240 min, 50 μl were withdrawn, diluted with 100 μl acetonitrile, vortexed for 15 sec and centrifuged (13000 rpm, 3 min). 5 μl of supernatant were injected for HPLC/UV analysis.

| column: | Acquity UPLC BEH C18, 50 × 2.1 mm i.d., dp = | |
|---|---|---|
| | 1.7 μm | |
| oven temperature: | 40° C. | |
| flow: | 0.5 ml/min | |
| solvent A: | Acetonitrile | |
| solvent B: | 20 mM KH₂PO₄, pH 4.25 | |
| gradient: | time [min] | solvent B [%] |
| | 0.00 | 40 |
| | 2.00 | 40 |
| | 6.00 | 15 |
| | 10.00 | 15 |
| | 10.10 | 40 |
| | 15.00 (stoptime) | 40 |

| | |
|---|---|
| detection: | DAD (λ = 248 nm (60 mm cell)) |

c) Quantification of Licofelone by HPLC/UV

A set of six reference solutions (solvent: 1 vol IF and 4 vol 2% bovine serum albumin in FaSSIF pH 6.5) with licofelone concentrations between 38.0 and 0.19 μg/ml and the solvent without licofelone were analyzed in duplicate. The obtained concentration/peak area data pairs were subjected to linear regression analysis and the resulting calibration curve (r²=0.9999) was used for quantification of licofelone in incubation experiments.

d) Results

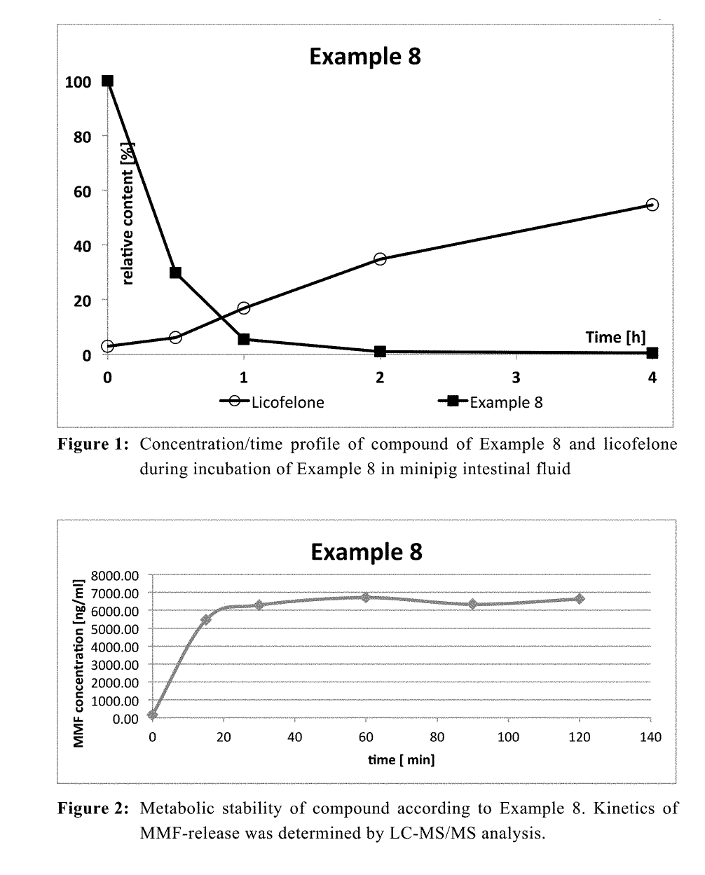

Incubation of NSAID/Licofelone compound as produced in Example 8 in diluted minipig intestinal fluid resulted in an unexpected smooth cleavage of the ester bond and thus in a release of the individual moieties, such as licofelone and monomethylfumarate. The analytical method allowed for quantification of the release of licofelone.

| Time | 0.5 h | 1 h | 2 h | 4 h | Conc./time profile |
|---|---|---|---|---|---|
| Concentration | 6.1 | 16.9 | 34.8 | 54.7 | FIG. 1 |
| licofelone [%] | | | | | |

### Example 10: Metabolic Stability of Prodrugs in Minipig Intestinal Fluid—Quantification of Monomethylfumarate

a) Incubation Experiments with Minipig Intestinal Fluid

In a HPLC glass vial, 8 μl of stock solution were mixed with 792 μl dil IF and the mixture was stirred (250 rpm) in a water bath (T=37° C.).

Immediately after mixing as well as at t=15 min, 30 min, 60 min, 90 min and 120 min, 50 μl were withdrawn and prepared for LC-MS analysis.

Incubations were continued and in case the result of analysis of the 120 min indicated the presence of remaining intact MMF prodrug, additional samples were taken (t=360 or 420 min and at 1,260 or 1,320 min) and analyzed.

b) Quantification of MMF by LC-MS

MMF in intestinal fluid was quantified by means of a validated LC-MS/MS method. Prior to the analysis of test samples, a calibration curve was established. Each calibration solution was analyzed two-fold. The second analysis was carried out approx. 18 h after storage of the sample in the autosampler, which was cooled to 8° C. The results demonstrate that the ratio of peak areas remains essentially unchanged between the first and the second analysis. The concentration/peak area ratio data pairs were subjected to regression analysis with 1/x weighting and the resulting calibration equation was used to quantify the MMF content in incubation samples. As Internal Standard (ISTD), monomethyl fumarate was used.

Analyses were developed and performed on an Acquity UPLC system coupled with a TQ detector (triple quadruple mass spectrometer).

Column: Phenomenex Kinetex C18, 100 A, 2.6 μm
(150×4.6 mm)

Flow: 0.4 ml/min

Split: appr. 100 μl/min to MS

Temperature: 30° C.
Solvent system (isocratic):
Solvent A 25% water with 0.1% acetic acid

Solvent B 75% methanol with 0.1% acetic acid

Stoptime: 6 min

Autosampler temperature: 8° C.

Injection volume: 4 μl

Retention time:

MMF: 4.3 min

DMF: 4.7 min

Mass Spectrometry

software: Masslynx 4.1

detection mode: electrospray/negative ions (ESP −)

capillary voltage: 2.3 kV

source temperature: 100° C.

desolvation temperature: 450° C.

cone voltage: 18 V

desolvation gas: N₂, 650 L/h

cone gas: N₂, 20 L/h

collision gas: argon, appr. 3.3*10⁻³ mbar

collision energy: 11 eV

MRM [m/z]: 128.94>85.03 Monomethylfumarate dwell: 200 msec
142.99>99.06 Monoethylfumarate (ISTD)
dwell: 200 msec

Sample Preparation

50 μl sample (calibration solution or sample of an incubation experiment with MMF prodrugs) was mixed with 50 μl WS_{ISTD}, 20 μl formic acid and 100 μl acetonitrile. This mixture was vortexed for 15 sec and centrifuged (13,000 rpm, 3 min). Thereafter, 4 μl of the supernatant were subjected to LC-MS analysis.

The release of MMF is shown in FIG. 2.

### Example 11: Investigation of the Effect of Test Compound in Experimental Encephalomyelitis in the Mouse

Assessment of the efficacy of compounds of the invention in MOG₃₅₋₅₅-induced experimental autoimmune encephalomyelitis (EAE) in C57BL/6 mice:
Test system: male C57BL/6 mice, 12 weeks old; 10 animals per treatment group;
Induction of EAE: Day-1—subcutaneous injection of MOG35-55, suspended in complete Freund's adjuvans and intraperitoneal injection of pertussis toxin.
   Day 3—intraperitoneal injection of pertussis toxin.
Treatment: Compounds according to Example 9 as test substances or vehicle only were administered via oral route. Test substances were dissolved or suspended in 0.5% hydroxyethylcellulose (dissolved in 50 mM potassium dihydrogenphosphate, pH 5.0). Drug concentration in dose formulations: 11.54 mM;
Dose volume: 10 ml/kg body weight;
Start of treatment: Day 1
Observations (clinical Observations were recorded daily between day 1 and
score and body weight): 13.
Clinical score: grade 0-10; 0 (=no impairments), 1 (normal movement; limp tail: proximal 2/3 of the tail is limp and droopy), 2 (normal movement; whole tail is limp; 3 (wobbly walk; absent righting reflex), 4 (gait ataxia), 5 (mild paraparesis), 6 (moderate paraparesis), 7 (severe paraparesis or paraplegia), 8 (tetraparesis), 9 (moribund), 10 (death).

Results:

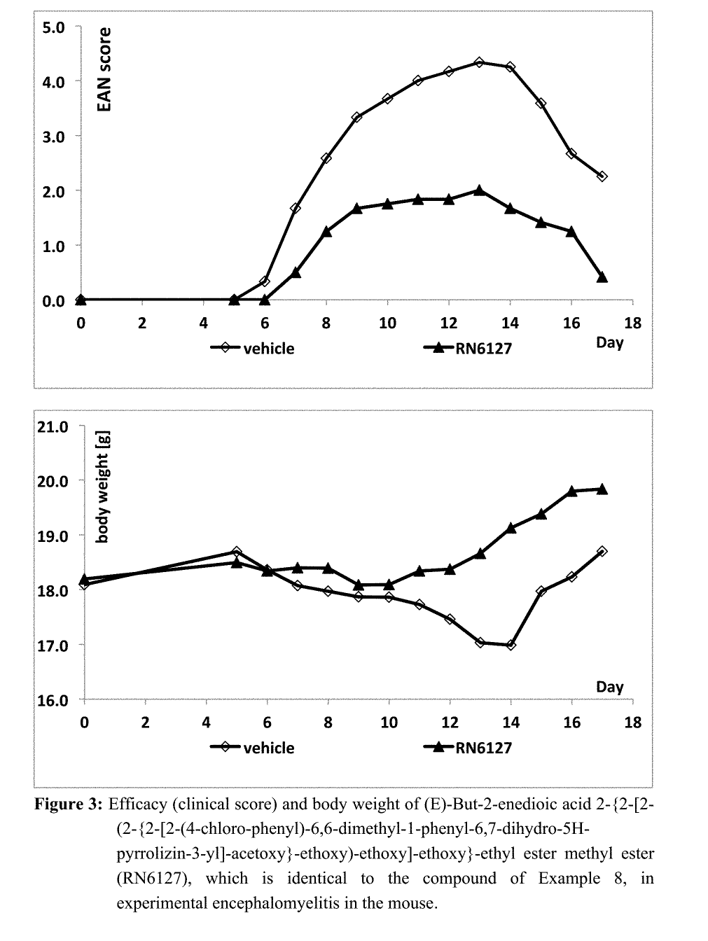

The results of the treatment with the compound according to Example 8 are shown in FIG. 3. The treatment was effective.

1. Compound according to Formula (I) 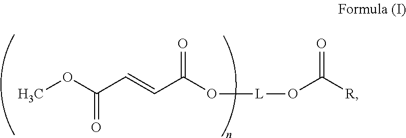
wherein
n is 1 or 2,
L is —((CH₂)₂O)ₘ—(CH₂)₂— wherein m is 1 to 5, and —OOCR is a carboxylate oft 2-(2,3-dimethylphenyl)aminobenzoic acid (mefenamic acid), 2-{[3-(trifluoromethyl)phenyl]amino}benzoic acid (flufenamic acid), 2-[(2,6-dichloro-3-methylphenyl)amino]benzoic acid (meclofenamic acid), 2-{[3-(trifluoromethyl)phenyl]amino}nicotinic acid (niflumic acid), 2-[2-[2-[(2,6-dichlorophenyl)amino]phenyl]acetyl]oxyacetic acid (aceclofenac), 2-[2-[1-(4-chlorobenzoyl)-5-methoxy-2-methylindol-3-yl]acetyl]oxyacetic acid (acemetacin), 2-(2-(2,6-dichlorophenylamino)phenyl)acetic acid (diclofenac), 2-(1,8-diethyl-4,9-dihydro-3H-pyrano[3,4-b]indol-1-yl)acetic acid (etodolac), 2-{1-[(4-chlorophenyl)carbonyl]-5-methoxy-2-methyl-1H-indol-3-yl}acetic acid (indometacin), 5-benzoyl-2,3-dihydro-1H-pyrrolizine-1-carboxylic acid (ketorolac), 2-[2-amino-3-(4-bromobenzoyl)phenyl]acetic acid (bromfenac), (RS)-2-(4-(2-methylpropyl)phenyl)propanoic acid (ibuprofen), (2S)-2-[4-(2-methylpropyl)phenyl]propanoic acid (dexibuprofen), (+)-(S)-2-(6-methoxynaphthalen-2-yl)propanoic acid (naproxen), (RS)-2-(3-benzoylphenyl)propanoic acid (ketoprofen), (2S)-2-[3-(benzoyl)phenyl]propanoic acid (desketoprofen), (RS)-2-(2-fluorobiphenyl-4-yl)propanoic acid (flurbiprofen), 2-[2-(4-chlorophenyl)-1,3-benzoxazol-5-yl]propanoic acid (benoxaprofen), (RS)-2-(5-benzoyl-2-thienyl)propanoic acid (tiaprofenic acid), or 2-(acetoxy)benzoic acid (aspirin) or [6-(4-chlorophenyl)-2,2-dimethyl-7-phenyl-2,3-dihydro-1H-pyrrolizin-5-yl]acetic acid (licofelone)
or a pharmaceutically acceptable salt, hydrate, solvate, polymorph and/or mixtures thereof.

2. Compound according to claim 1, wherein m is 3.

3. Compound according to claim 1 or 2 for use as a medicament.

4. Compound according to any one of the preceding claims for use in the treatment of systemic diseases, autoimmune diseases or inflammatory diseases, preferably for the use in the treatment of osteoarthritis, psoriatic arthritis, reactive arthritis, migraine, (acute) gout, metastatic bone pain, muscle stiffness and pain due to Parkinson's disease, ileus and renal colic and/or multiple sclerosis, rheumatoid arthritis or psoriasis.

5. Pharmaceutical composition comprising a compound according to any one of the preceding claims.

6. Pharmaceutical composition according to claim 5, comprising
(i) 0.01 to 10 mmol of a compound according to claim 1 or 2 and
(ii) optionally pharmaceutical excipients.

7. Pharmaceutical composition according to claim 5 or 6, wherein the composition is a solid oral dosage form.

8. Method for treating and/or preventing systemic diseases, autoimmune diseases and/or inflammatory diseases, preferably osteoarthritis, psoriatic arthritis, reactive arthritis, migraine, (acute) gout, metastatic bone pain, muscle stiffness and pain due to Parkinson's disease, ileus and renal colic and/or multiple sclerosis, rheumatoid arthritis, or psoriasis, in particular multiple sclerosis, comprising administering to a subject in need thereof a therapeutically effective amount of the compound according to any one of claims 1 to 4 or the pharmaceutical composition according to any one of claims 5 to 7.
